(19) United States
(12) Patent Application Publication
(10) Pub. No.: US 2025/0222141 A1
(43) Pub. Date: Jul. 10, 2025

(54) COMPOSITION FOR THE DETECTION AND PARTIAL DECONTAMINATION OF CHEMICAL THREAT AGENTS ON SKIN SURFACE FOLLOWING DERMAL EXPOSURE

(71) Applicant: THE UNITED STATES GOVERNMENT, AS REPRESENTED BY THE SECRETARY OF THE ARMY, Fort Detrick, MD (US)

(72) Inventors: Shane Aaron KASTEN, Fallston, MD (US); Zachary Andrew CANTER, Pylesville, MD (US)

(21) Appl. No.: 18/865,872

(22) PCT Filed: May 16, 2023
(86) PCT No.: PCT/US2023/022368

§ 371 Date: Jan. 23, 2025

Related U.S. Application Data
(60) Provisional application No. 63/364,728, filed on May 16, 2022.

(51) Int. Cl. A61K 49/00; A61B 5/00; A61K 9/00; A61K 9/70; A61K 38/39; A61P 39/00

(52) U.S. Cl. CPC A61K 49/0056; A61K 9/0014; A61K 9/70; A61K 38/39; A61K 49/0073; A61P 39/00; A61B 5/0071

(57) ABSTRACT

The invention entails methods to detect and at least partially decontaminate an intact skin surface of a person suspected of exposure to harmful substance, such as cholinesterase inhibitors (i.e., nerve agents, carbamate pesticides), vesicants or synthetic opioids. The method uses a two-step film-forming composition, which indicates dermal contact of harmful substances, and at least partially decontaminate the skin surface if needed.

### BACKGROUND

Most chemical agents designed for warfare or poisonings are extremely potent substances, often devoid of color and/or odor. A dermal exposure to a persistent hazardous or toxic substance can happen unknowingly. At this time, identifying the exact site of exposure can be nearly impossible without involving off-the-body analytical technologies found in a specialized laboratory. The inability to rapidly identify the site of exposure requires blinded spot decontamination that can antagonize the exposure. An exposed individual will likely incur injury followed by potential lethal consequences if removal and/or proper decontamination of the toxic substance is not conducted in a timely manner.

### SUMMARY

This invention provides a rapid, visual indication of dermal contact to specific toxic chemical substances to assist in decontamination efforts in an austere environment.

This invention contemplates a method to (1) indicate dermal contact of toxic chemical substances, (2) guide stringent decontamination, and (3) at least partially decontaminate the skin surface if needed. Preferably, the method uses a two-step application of a film-forming composition (e.g., a film-forming spray). The invention also contemplates the unique formulations in each step of the film-forming composition. The invention also contemplates a kit that includes all components needed for the two-step method, including optional instructions, which can be useful in austere conditions.

A specific application of this invention would be its use in response to a suspected dermal exposure to a cholinesterase (ChE) inhibiting substance (i.e., nerve agent or organophosphorus or carbamate pesticides). These substances are extremely potent, colorless, and odorless. A few micro-liters of neat nerve agents can be nearly impossible to locate on the skin's surface and absolutely lethal if left untreated. Recent world events in which nerve agent was used to poison individuals by dermal exposure compel the need for this invention. The utility of this invention can be easily imagined when considering the poisoning of the Skripal family (Salisbury, UK) in 2018 in which a door knob was tainted with the extremely toxic Novichok nerve agent, or the death of Dawn Sturgess who died after unknowingly spraying herself with perfume containing Novichok sending her into cardiac arrest.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1 shows a general diagram of the detection of harmful substances according to disclosed embodiments.

FIG. 2 shows a general diagram of a method embodiment using a two-part spray method for detection of a harmful agent on exposed on a subject's surface.

FIG. 3 shows a small portion of porcine belly skin exposed to a ChE inhibitor (nerve agent surrogate) at two different sites on the surface of the skin. Ten minutes after the inhibitor was spotted to the skin, Spray A was sprayed over the skin surface within 5 seconds. Spray B was then sprayed over 7 seconds to cover the exact surface that Spray A covered with exposure sites becoming apparent during application. After 25 seconds of technology application, the exposure sites are clearly visible as the background color continues to fade. At this point the two sprays have combined to become a soft hydrogel over the surface of the skin. This hydrogel reduces the spread of the exposure, limits bleed/dilution of the color indication, and becomes a sponge that traps the ChE inhibitor away from the skin resulting in physical removal and thus decontamination. At 30 seconds post-application, a credit card is used to scrape the hydrogel spots from the surface of the skin allowing removal of the exposing substance and deposition into a test tube for lab analysis.

FIG. 4. is an in vitro demonstration of the 2-part spray filming technology detecting 6 different persistent nerve agents (i.e., VX) on a plastic surface along with carrier-solvent controls to show that exposure site detection is dependent on the presence of nerve agent. Nerve agents were spotted in triplicate. Blue spots emerge within seconds of application of Spray B as the background color fades from purple to pink rapidly (30 s). Three different UV flashlights are shown in the middle-bottom panel. In dim lighting, a couple spots are illuminated by on of the UV flashlight showing the blue spot turns red due to its optical activity.

FIG. 5 illustrates detection of 6 different persistent nerve agents applied to various locations on a piece of excised porcine skin. 50 μL of each agent (diluted ˜1000× from neat concentration in volatile organic solvent, representing 35-50 μg neat agent) was pipetted onto the surface and the solvent allowed to evaporate. An equivalent volume of solvent was also added to control for solvent effects as indicated. Immediately after application of the invention (˜10 s, top middle), exposure sites are evident and after 30 s (top right), exposure sites are clearly visible. After 60 s, (bottom left) the background is nearly colorless. Exposure spots are still visible under low light conditions (bottom middle) and give bright red/pink fluorescence (bottom right) when illuminated with UV light.

FIG. 6 illustrates 2 spots of ChE inhibitor surrogate spotted onto a plastic surface with the invention applied (left). The lower spot is illuminated with UV radiation in low light conditions (middle) displaying red/pink fluorescence. The lower spot is also illuminated with green light (510-540 nm) and observed through a 600 nm longpass red filter lens (right) displaying ultra-bright red fluorescence

FIG. 7 shows the structures of sulfur mustard (left top) and CEES (left bottom). CEES is a less-toxic sulfur mustard simulant colloquially known as “half-mustard.” Neat CEES was spotted onto a piece of excised porcine skin in triplicate at 1, 2, 5, 10, and 20 μL and the invention embedded with the vesicant reagent chemistry was applied and visualized with UV light in low light conditions after 3 minutes (middle). These spots are easily visible as indicated by the bright blue spots. It should be noted that the 20 μL spots ran together and triplicates appear as a single large spot at the bottom of the surface. The smallest volume applied, 1 μL, can be easily visualized under normal lighting conditions 6 minutes after spray film application (right).

FIG. 8 shows the chemical structure of fentanyl (left). Fentanyl (0.25, 0.5, 1, 2 and 4 mg) was spotted onto a piece of excised porcine skin in duplicate and the invention embedded with the synthetic opioid reagent chemistry was applied. Approximately 10s after application, spots indicating the precise location of the fentanyl is evidenced by pink spots under normal lighting conditions (middle). These spots are also easily seen as dark purple spots on a fluorescent background when viewed under UV light with dim ambient lighting (right).

FIG. 9 is a photo of a prefabricated elastomeric film with reagents impregnated therein for detection of a harmful substance.

FIG. 10 provides a photograph showing proof-of principle prototype applied to gloved hands in the laboratory setting, on the left (blue film). In the picture on the right, the hand on the left had vehicle/solvent dried on the back of the hand to serve as a negative control. On the hand on the right, a surrogate substance that simulates the effects of microliter amounts of ChE inhibitor was applied. Activating spray was applied to both hands followed by application of the premade blue REF. Within three minutes a distinct blue spot can be clearly identified exactly where the surrogate substance was dried on the back of the hand while the negative control gave no indication as evidenced by complete absence of blue color.

FIG. 11. 20 μL of a ChE inhibitor surrogate in IPA (125 mg/mL) was applied to quadrant II of a hairless guinea pig along with an equal volume of IPA in each remaining quadrant. Pictures are shown A. before application (t=−30 s) B. after application of Spray A (t=−20 s) C. after application of Spray B (t=0 s) D. 10 seconds after full spray application E. 1.5 minutes after spray application F. Scraping of unexposed quadrant IV G. Spray under UV light, note the bright pink spot indicating exposure H. Spray under green light with a red filter, note the bright spot indicating exposure and I. Removing of the exposure site

FIG. 12: Spray film was reapplied to the animal shown in FIG. 11 and pictures above show A. Spray A applied (t=−25 s) B. Spray B applied (t=−20 s) C. 20 seconds after full application of the spray film D. Scraping of the exposed quadrant E. 2^{nd} reapplication of Spray A F. 2 reapplication of Spray B G. 20 s after 2^{nd} reapplication of Spray B and H. removal of the 2^{nd} reapplication. Note that the intensity of the blue spot indicating exposure site has decreased from the original spray in FIG. 1 through the 2^{nd} reapplication of the spray.

FIG. 13 VX in IPA was applied to quadrant IV of a hairless guinea pig along with an equal volume of IPA in each remaining quadrant. Pictures are shown A. before application (t=−35 s) B. after application of Spray A (t=−25 s) C. after application of Spray B (t=0 s) D. 10 seconds after full spray application E. After removal of spray film in the exposed quadrant F. After removal of all quadrants.

FIG. 14 provides a diagram showing the chemistry involved in detecting ChE inhibitors.

### DETAILED DESCRIPTION

### Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

The term “ionic liquid” as used herein refers to a salt that is found the liquid state at room temperature and pressure. While ordinary liquids such as water and gasoline are predominantly made of electrically neutral molecules, ionic liquids are compounds made completely of ions. Examples of ionic liquids include but are not limited to 1-Ethyl-3-methylimidazolium dicyanamide (EMIM DCA), 1-Allyl-3-methylimidazolium dicyanamide (AMIM DCA) and 1-Allyl-3-methylimidazolium bis(trifluoromethylsulfonyl)imide (AMIM TFSI).

The term “reactive agent” as used herein refers to an agent that is part of a reaction, or a series of reactions, with a suspected harmful substance that produces a visually detectable reaction product.

The term “visually detectable reaction product” as used herein refers to product that is detectable under different forms of light including white light, green light, or ultraviolet light.

The term “harmful substance” as used herein refers to a chemical substance that causes an adverse and potentially fatal effect in a subject.

As used herein, the terms “subject,” “individual,” “host,” and “patient,” are used interchangeably to refer to any animal, and can include humans, simians, avians, felines, canines, equines, rodents, bovines, porcines, ovines, caprines, mammalian farm animals, mammalian sport animals, and mammalian pets. A preferred subject is a human patient.

The term “manual pump mechanism” as used herein refers to a pump that is able to propel a substance out of a container without the use of a propellant. In an example, a manual pump mechanism comprises an actuator that upon being depressed sprays a substance out of a nozzle.

The term “liquid medium” as used herein refers to a carrier into which chemical components can be suspended and/or dissolved. In an example, a liquid medium pertains to water, with optional buffer or pH modifying agents.

The term cholinesterase (ChE) inhibitor refers to chemical compounds that are inhibitors of cholinesterase enzymes such as acetylcholinesterase (AChE) and butyrylcholinesterase (BChE). Examples include the nerve agents sarin, VX, and Novichok as well as organophosphorus and carbamate pesticides.

The term “cold-water fish gelatin” or “CWFG” refers to a gelatin produced from any species of fish living predominantly in water of 18° C. or below, i.e. cold-water fish. Examples of cold-water fish include cod, haddock, hake, pollock, cusk, sole, flounder, turbot, halibut, plaice, lump fish, pike or salmon). CWFG generally have gel points below 15° C., typically 4 to 12° C., and melting points below 22° C., typically 12 to 19° C.

The term “hydrazide modified” as used herein refers to a treatment of gelatin with a hydrazide such as adipic acid dihydrazide (ADH) or carbohydrazide and amine-to-amide coupling reagents (such as hydroxybenzotriazole [HOBt], and 1-Ethyl-3-(3-dimethylaminopropyl)carbodiimide [EDC]) wherein the hydrazide interacts with a carboxyl groups of the glutamic acid side groups of the gelatin. See Hozumi et al., Biomolecules, 2018, 19:288-297.

The term “monoaldehyde modified” as used herein refers to a treatment of polysaccharide polymer with an agent such as (±)-3-Amino-1,2-propanediol (AP), amine-to-amide coupling reagents (such as hydroxybenzotriazole [HOBt], and 1-Ethyl-3-(3-dimethylaminopropyl)carbodiimide [EDC]) resulting in the in conversion of the carboxylic acid groups to diol groups. This conversion is followed by a reduction of the diol to an aldehyde functional group via reducing agent such as sodium periodate. See Hozumi et al. supra.

The term “opioid” as used herein refers to heroin, oxycodone and synthetic opioids such as fentanyl and carfentanil.

The term vesicant as used herein refers to sulfur mustards, nitrogen mustards, their analogues and breakdown products such as CEES (a.k.a half-mustard) and other structurally related agents that exert their toxic effects through alkylation. These agents may also be colloquially referred to as “blister agents.”

### OVERVIEW

In the Summary above, in the Detailed Description, and the claims below, as well as the accompanying figures, reference is made to particular features of the invention. It is to be understood that the disclosure of the invention in this specification includes all possible combinations of such particular features. For example, where a particular feature is disclosed in the context of a particular embodiment or embodiment of the invention, or a particular claim, that feature can also be used, to the extent possible, in combination with and/or in the context of other particular embodiments and embodiments of the invention, and in the invention generally. For the purposes of explanation, numerous specific details are set forth in order to provide a thorough understanding of the present invention. It will be apparent, however, to one skilled in the art that the present invention may be practiced without these specific details.

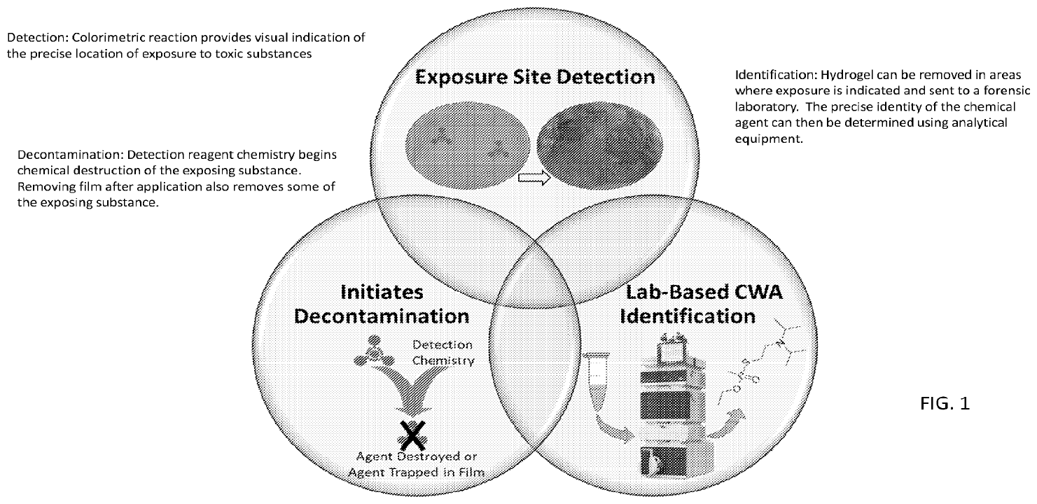

In one embodiment, this invention will be applied to a suspected exposure site to rapidly identify and support judicious decontamination such that inadvertent spread can be minimized. A first composition that is pre-reactive or pre-activating is either applied to or generated on the surface of the suspected exposure site is the key component. This is followed by with an activating and/or reactive and/or enhancing second composition. The first composition may be in any suitable form, preferably a film or a spray. The second composition may also be in any suitable form, preferably a spray. The second composition can be applied simultaneously, immediately after the first composition, or within a specified timeframe. FIG. 1 provides a diagram illustrating the underlying approach of typical embodiments described herein.

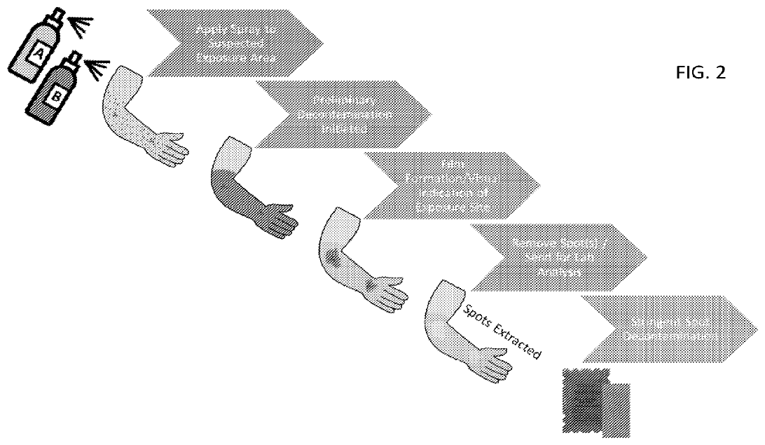

Typically, reagents responsible for activating the composition for hydrogel formation and/or for visually detecting a reaction product are at least partially separated and dispensed as two different sprays depending on the reagents involved and the harmful substance being detected. Some components cannot come into contact with the others prior to spraying. For example, a dye composition such as a blue dye may be included into Spray B due assist in visually determining where the user has coated over Spray A due to the color. FIG. 2 shows a diagram of a general approach of using a two-part spray system to detect presence of a harmful substance on a subject's skin. Also shown is a final optional step of applying a decontamination after the harmful substance is detected and the film capturing the harmful substance is removed. Embodiments of the invention will provide exact visual indication of exposure within a few minutes of application and may also assist in the decontamination process by absorption and subsequent physical removal of the peelable elastomeric film.

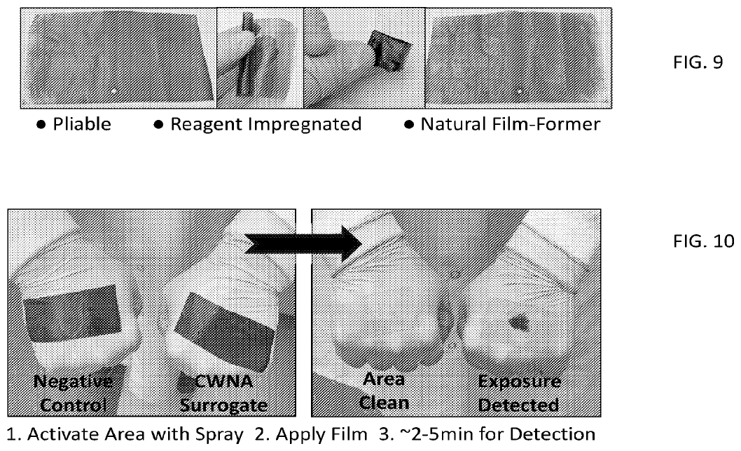

Preferably, both the first composition (reactive composition) and second composition (activating or enhancing composition) are in spray form. Another preferred form is that the first composition is a spray, and the second composition is a film. In the film embodiment, a separate spray composition would be used to activate and pre-moisten the area before the elastomeric film is applied. Once the film becomes wet it becomes sticky and thus welds down to the skin surface allowing exact exposure site detection without moving. The embodiment involving a prefabricated film is illustrated in FIGS. 9 and 10.

One skilled in the art will appreciate that the first composition and second composition can be stored and dispensed using known pump actuated containers. Some embodiments may involve dispensing from two separate pump actuated containers, or alternatively, a unitary dual-spray head container may be utilized so as to dispense the first and second compositions contemporaneously.

In one preferred embodiment, the first composition and second composition are in spray form. In this form, there is contemplated a skin disclosure spray for the site identification of exposure to facilitate stringent decontamination and/or assurance of sanitization. The two part spray system can detect sub-lethal levels of harmful substances on the surface of skin in seconds. The 2-part spray indicates exposure, holds the location of exposure via film formation, and is easily removed to expedite site decontamination and lab-analysis. Special optics can also be utilized to enhance detection via fluorescence when higher contrast is required. This medically-inert disclosure spray can also be used to detect and guide decontamination of exposure on material surfaces besides skin.

The advantages and benefits of this invention include:
Harmless and Rapid identification of sub-lethal dermal exposure site(s) Guides stringent decontamination of exposure site(s)
High color contrast for colorimetric and enhanced fluorogenic detection Reduces potential collateral contamination via instant film formation
Initiates decontamination process, (bio)chemically and by physical removal
facilitates lab-based identification; determine identity of the exposing substance using analytical methodologies/technologies
Assurance of sanitization via reapplication following decontamination

Certain embodiments described herein can be utilized at all roles of care on/off the austere battlefield to detect and guide stringent decontamination of skin or other material surfaces following exposure to a ChE inhibitor. Known or presumptive exposures can be rapidly detected on individuals showing declined blood ChE activity, those displaying overt signs/symptoms of exposure, those needing assurance of sanitization, or those desiring confidence of non-exposure. Embodiments could also indicate site(s) of exposure on deceased individuals. The disclosed embodiments will allow low-burden/complexity, rapid detection without need of analytical instrumentation. Aids in the reduction of recurring medical countermeasure administration for prolonged treatment on/off the battlefield. Also contemplated are uses for detection of harmful substances in non-warfare scenarios.

### Detailed Description of Exemplary Embodiments

According to one embodiment, disclosed is a method to detect and at least partially decontaminate a surface of a subject suspected of being exposed to a harmful substance. The method involves
a) Applying to the surface a first composition comprising gelatin or a modified gelatin derivative such as adipic dihydrazide modified cold-water fish gelatin (CWFG-ADH); and
b) Applying to the surface a second composition comprising a modified polysaccharide polymer such as mono-aldehyde modified polygalacturonic acid (PGA-mCHO)
   Steps a) and b) form or deliver a film on the surface. The first composition or second composition may include at least one reactive agent that reacts with the suspected harmful substance to produce a visually detectable reaction product.

Those skilled in the art will appreciate that gelatin may include but is not limited to porcine gelatin, bovine gelatin, or cold-fish gelatin, and the like. Those skilled in the art will appreciate that the polysaccharide polymer can include, but is not limited to, alginates, carrageenans, carboxylated starches, carboxy-(C₁-C₆-alkyl) cellulose, gellans, hyaluronic acid, pectins, and xanthans. A modified gelatin derivative is a gelatin that has been modified that can react with a modified polysaccharide polymer according to a Schiff base reaction. In a specific embodiment, the gelatin is hydrazide modified and the polysaccharide polymer is PGA that has been monoaldehyde modified whereby the modified gelatin derivative and modified PGA cross-link rapidly according to a Schiff base reaction to produce a hydrogel.

In one specific embodiment, the suspected harmful substance pertains to a ChE inhibitor. The ChE inhibitor may include but is not limited sarin, VX, or Novichok. In a specific embodiment, the first composition comprises BChE that is inactivated/inhibited by ChE inhibitor. The second composition comprises an R-TCh that is hydrolyzed by any uninhibited BChE yielding TCh which subsequently reacts with resazurin or other suitable indicator dye initiating a color change. Visual indication of presence of ChE inhibitor may be given by a persistent colored spot (i.e., no hydrolysis of the R-TCh and no reaction with resazurin). Visual indication of the absence of ChE inhibitor may include conversion of blue coloration to pink followed by disappearance of color. In an alternative embodiment, acetylcholinesterase (AChE) can be used in place of BChE. The visually detectable reaction product may include a visually detectable pink color under UV light.

According to another embodiment, the suspected harmful substance comprises a vesicant agent and the at least one reactive agent comprises luminol and an ionic liquid. Further, the visually detectable reaction product may possess fluorescence.

According to another embodiment, the harmful substance is a synthetic opioid and at least one reactive agent comprises Eosin Y. The Eosin Y can complex with a tertiary amine of the synthetic opioid to form the visually detectable reaction product. Visual indication of reaction product may be given by a dark purple spot under ambient lighting. Visual indication of reaction product may also be given by absence of fluorescence under UV light.

The above described method variations may further involve removing at least a portion of the film from the surface in which the visually detectable reaction product is produced. In addition, the method variations may further involve analyzing the removed portion of the film to confirm identify of the harmful substance, such as by standard chemical testing methods known in the art.

As mentioned above, delivering at least one reactive agent to the suspected harmful substance may also act to at least partially neutralize the harmful substance. In alternative embodiments, the first and/or second compositions may further include one or more decontamination enhancing agents, such as a catalyst for destroying the exposing agent (which may have an additive effect to decontamination in conjunction with the at least one reactive agent) or a trapping agent to sequester the agent into the hydrogel after it forms to aid in physical removal. Examples of catalysts include zirconium hydroxide, metallo-organic frameworks (MOFs) and the like. Trapping agents may include HLB-nano/micro particles for high affinity binding or silica nano/micro particles or cationic/anionic nano/micro particles.

According to another specific method embodiment, disclosed is a method to detect and at least partially decontaminate a surface of a subject suspected of being exposed to a ChE inhibitor, vesicant or synthetic opioid. The steps of this method include
(a) Applying to the surface a film-forming reactive composition that visually indicates exposure to a ChE inhibitor, vesicant, or synthetic opioid,
(b) Applying to the surface an activating composition that reacts with the reactive composition, wherein exposure to ChE inhibitor is rapidly detected, the film absorbs at least some of the ChE inhibitor, and film is peelable from the skin surface.

For the above method embodiments, the visually detectable reaction product develops in less than 5 minutes. In a preferred embodiment, the visually detectable reaction product develops in less than 1 minute.

In another embodiment, disclosed is a system for detecting and at least partially decontaminating a surface of a subject suspected of being exposed to a harmful substance, the system comprising
a) a first composition comprising adipic dihydrazide modified cold-water fish gelatin (CWFG-ADH); and
b) a second composition comprising mono-aldehyde modified polygalacturonic acid (PGA-mCHO) and/or pectic acid; and
   wherein the first composition and/or second composition comprises at least one reactive agent.

The above system may further include a first container in which the first composition is disposed and a second container in which the second composition is disposed. In an alternative version, the first composition and/or the second composition are in a reconstitutable form, and the system further includes at least one liquid medium into which the first and/or second composition can be reconstituted.

In a specific embodiment of the above system, the suspected harmful substance comprises a ChE inhibitor. The ChE inhibitor may include but is not limited to sarin, VX or, Novichok, and the first composition includes a butyrylcholinesterase enzyme (BChE) and the second composition comprises resazurin. The second composition may further comprise acetylthiocholine (ATCh) chloride or other thiocholine containing substrates. In a more specific embodiment, the first composition comprises butyrylcholinesterase enzyme at a concentration of about 0.50 mg/mL to about 1.0 mg/mL, CWFG-ADH at about 2 to about 10% w/v, and CaCl2) at about 5 to about 15 mM and the second composition comprises PGA-mCHO at about 1% to about 6% w/v, resazurin at about 0.1 mg/mL to about 1.0 mg/mL resazurin and ATCh chloride at about 25 mM to about 75 mM.

According to another specific embodiment of the system, the suspected harmful substance comprises a vesicant agent and the at least one reactive agent comprises luminol and an ionic liquid. Typically, the luminol and ionic liquid are in the second composition. Where the harmful substance is a vesicant, a specific embodiment involves the first composition comprising CWFG-ADH at about 2 to about 10% w/v, and CaCl2) at about 5 to about 15 mM and the second composition comprising PGA-mCHO at about 1% to about 6% w/v, luminol sodium salt at about 0.1 mM to about 0.9 mM and ionic liquid at about 0.5M to about 1.5 M.

According to another specific embodiment of the system, the harmful substance is a synthetic opioid and the at least one reactive agent comprises Eosin Y. Typically, the at least one reactive agent is in the second composition. When the harmful substance is a synthetic opioid, a specific embodiment involves the first composition comprising CWFG-ADH at about 2 to about 10% w/v, and CaCl2) at about 5 to about 15 mM and the second composition comprising PGA-mCHO at about 1% to about 6% w/v, Eosin Y at about 100 microM to about 500 microM.

The first container and/or second container may comprise a manual pump mechanism. In an alternative embodiment, either the first container and/or second container includes a propellent.

In an alternative system embodiment, the first composition is loaded onto a prefabricated elastomeric film. The elastomeric film may be adhered upon the skin of the subject after application of the second composition to the film or to the skin of the subject.

The first composition of the system may further include water and CaCl₂), or alternatively CuCl₂ or MgCl₂, and the second composition comprises water. The first and or second compositions may also include glycerol.

In another embodiment, disclosed is a kit comprising a first container and second container. The first and second container contain a first and second compositions, respectively as described above.

In an alternative embodiment of the kit, the first composition and second composition are in reconstitutable form. In such embodiment, the kit will also typically include a third container comprising a liquid medium comprising CaCl₂) and water, and optionally, a fourth container comprising a liquid medium comprising water and no CaCl₂).

### EXAMPLES

### Example 1: Specific Formula Embodiment for Detection and Partial Decontamination of ChE Inhibitors (i.e., Nerve Agents, Carbamate Pesticides)

| | [F] (final | | |
|---|---|---|---|
| | concentration) | | Purpose |
| Spray A | | | |
| Butyrylcholinesterase | 0.75 | mg/mL | Hydrolyzes ATCh when not in the presence |
| Enzyme (human) | | | of ChE inhibitor. Could be substituted |
| | | | with Acetylcholinesterase. |
| GEL-ADH (pH = 7 | 6% (w/v) | | Film forming component |
| by NaOH) | | | |
| CaCl₂ | 10 | mM | Aids rapid film forming by causing |
| | | | condensation of the PGA-mCHO |
| | polymer | | |
| Glycerol | 0.1% | | Plasticizer |
| H₂O | — | | |
| Spray B | | | |
| Resazurin | 0.4 | mg/ml | Redox Reporter Dye |
| Glycerol | 0.1% | | Plasticizer |
| Acetylthiocholine | 50 | mM | Substrate for BChE/Product reacts with Rez to |

| | | |
|---|---|---|
| (ATCh) Chloride | | cause color change |
| PGA-mCHO (pH = 7 | 4% (w/v) | Film forming component |
| by NaOH) | | |
| H2O | — | |

### Example 2: Specific Formula Embodiment for Detection of Vesicants

| | [F] (final | |
|---|---|---|
| | concentration) | Purpose |
| Spray A | | |
| GEL-ADH (pH = 7 by | 6% (w/v) | Film forming component |
| NaOH ) | | |
| CaCl₂ | 10 mM | Aids rapid film forming by |
| | | causing condensation of |
| | | the PGA-mCHO polymer |
| Glycerol | 0.1% | Plasticizer |
| H₂O | — | |
| Spray B | | |
| Luminol Sodium Salt | 0.55 mM | Reporter Dye |
| Glycerol | 0.1% | Plasticizer |
| 1-Ethyl-3- | 1.2M | Ionic Liquid (polarizing |
| methylimidazolium | | solvent that provides |
| dicyanamide (EMIM | | the requisite chemical |
| DCA) | | milieu for alkylation |
| | | by the alkylating agent |
| PGA-mCHO (pH = 7 by | 4% (w/v) | Film forming component |
| NaOH) | | |
| H2O | — | |

See Kumar et al., Anal Chem, 2021, 93: 1193-1199.

### Example 3: Specific Formula Embodiment for Detection of Opioids

| | [F] (final | |
|---|---|---|
| | concentration) | Purpose |
| Spray A | | |
| GEL-ADH (pH = 7 by | 6% (w/v) | Film forming component |
| NaOH) | | |
| CaCl₂ | 10 mM | Aids rapid film forming by |
| | | causing condensation of the |
| | | PGA-mCHO polymer |
| Glycerol | 0.1% | Plasticizer |
| H₂O | — | |
| Spray B | | |
| Eosin Y | 300 microM | Reporter Dye |
| Glycerol | 0.1% | Plasticizer |
| PGA-mCHO (pH = 7 by | 4% (w/v) | Film forming |
| NaOH) | | component |
| H2O | — | |

See Canfield et al. J. Forensic Sci, 2020, 65: 1432-1442

### Example 4 In Situ Detection of ChE Inhibitors

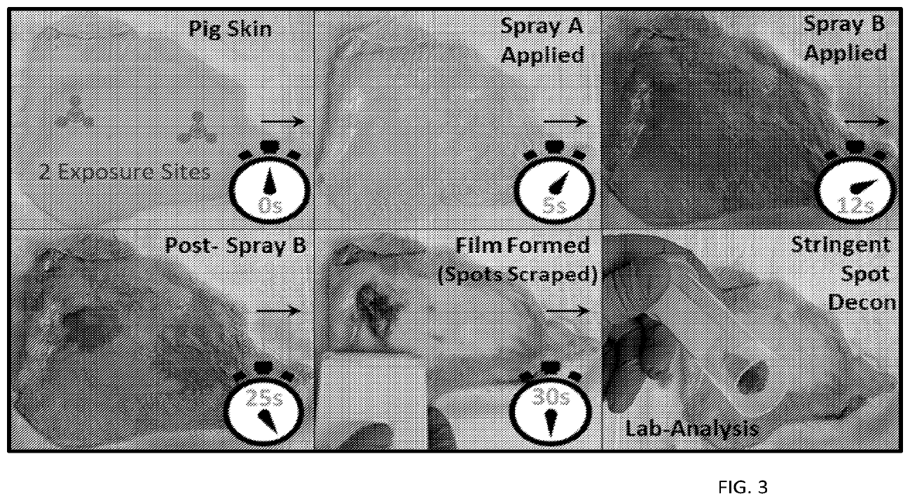

FIG. 3 shows a small portion of porcine belly skin exposed to a ChE inhibitor (nerve agent surrogate) at two different sites on the surface of the skin. Ten minutes after the inhibitor was spotted to the skin, Spray A was sprayed over the skin surface within 5 seconds. Spray B was then sprayed over 7 seconds to cover the exact surface that Spray A covered with exposure sites becoming apparent during application. After 25 seconds of technology application, the exposure sites are clearly visible as the background color continues to fade. At this point the two sprays have combined to become a soft hydrogel over the surface of the skin. This hydrogel reduces the spread of the exposure, limits bleed/dilution of the color indication, and becomes a sponge that traps the ChE inhibitor away from the skin resulting in physical removal and thus decontamination. At 30 seconds post-application, a credit card is used to scrape the hydrogel spots from the surface of the skin allowing removal of the exposing substance and deposition into a test tube for lab analysis.

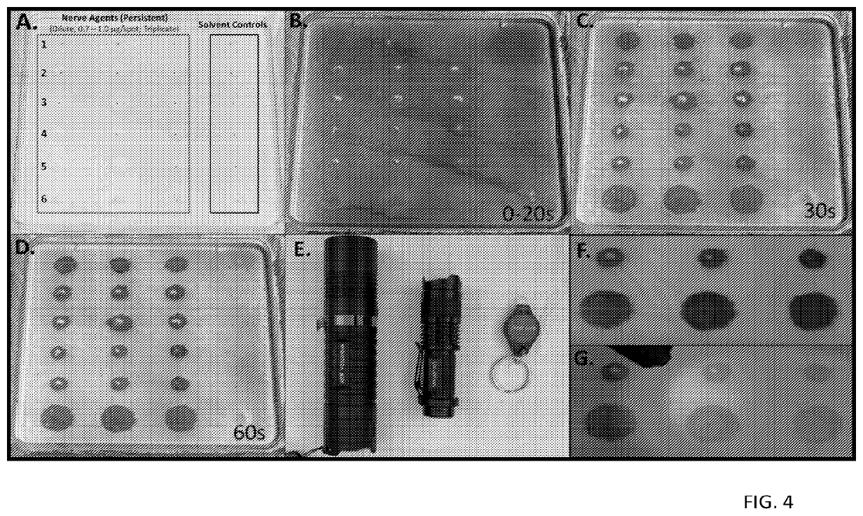

FIG. 4. provide figures showing a in vitro demonstration of the 2-part spray filming technology detecting 6 different persistent nerve agents (i.e., VX) on a plastic surface along with carrier-solvent controls to show that exposure site detection is dependent on the presence of nerve agent. Nerve agents were spotted in triplicate. Blue spots emerge within seconds of application of Spray B as the background color fades from purple to pink rapidly (30 s). Three different UV flashlights are shown in the middle-bottom panel. In dim lighting, a couple spots are illuminated by on of the UV flashlight showing the blue spot turns red due to its optical activity.

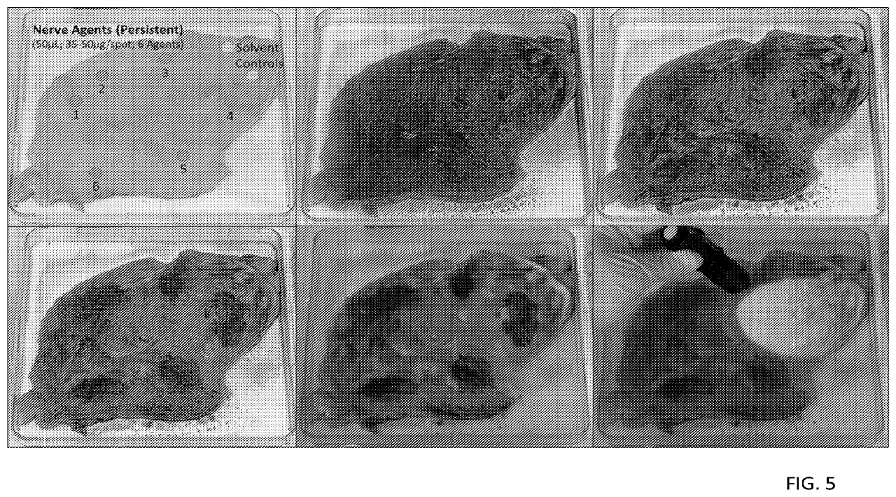

FIG. 5. illustrates detection of 6 different persistent nerve agents applied to various locations on a piece of excised porcine skin. 50 μL of each agent (diluted ˜1000× from neat concentration in volatile organic solvent, representing 35-50 μg neat agent) was pipetted onto the surface and the solvent allowed to evaporate. An equivalent volume of solvent was also added to control for solvent effects as indicated. Immediately after application of the invention (˜10 s, top middle), exposure sites are evident and after 30 s (top right), exposure sites are clearly visible. After 60 s, (bottom left) the background is nearly colorless. Exposure spots are still visible under low light conditions (bottom middle), and give bright red/pink fluorescence (bottom right) when illuminated with UV light.

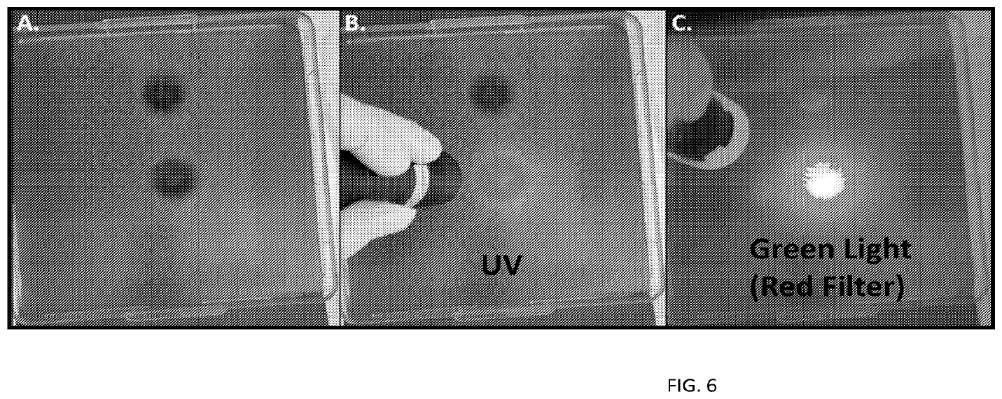

FIG. 6 illustrates 2 spots of ChE inhibitor surrogate spotted onto a plastic surface with the invention applied (left). The lower spot is illuminated with UV radiation in low light conditions (middle) displaying red/pink fluorescence. The lower spot is also illuminated with green light (510-540 nm) and observed through a 600 nm longpass red filter lens (right) displaying ultra-bright red fluorescence

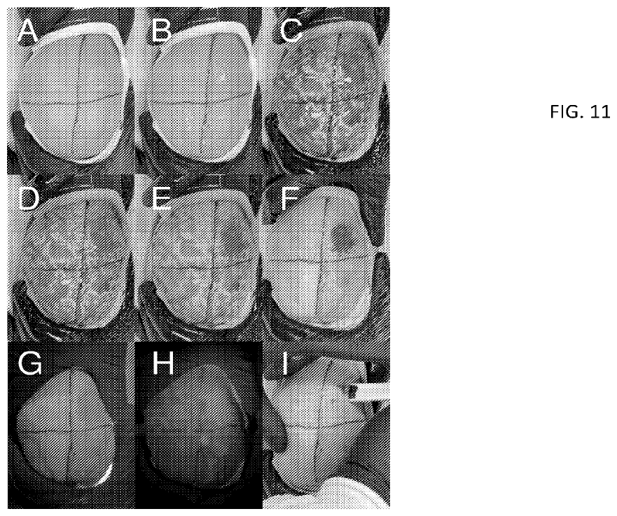

FIG. 11 illustrates how system embodiments comprising a first and second composition can detect the presence of a ChE inhibitor on guinea pig skin. According to this example, 20 uL of ChE inhibitor in IPA (125 mg/mL) was applied to quadrant II of a hairless guinea pig along with an equal volume of IPA in each remaining quadrant. Pictures are shown A. before application (t=−30s) B. after application of Spray A (t=−20 s) C. after application of Spray B (t=0 s) D. 10 seconds after full spray application E. 1.5 minutes after spray application F. Scraping of unexposed quadrant IV G. Spray under UV light, note the bright pink spot indicating exposure H. Spray under green light with a red filter, note the bright spot indicating exposure and I. Removing of the exposure site. As shown in FIG. 11, application of the first composition (spray A) and second composition (spray B) of Example 1 produces a clearly detectable blue spot on the skin with minimal background (FIG. 11F). Further, under Uv light, the detectable spot is shown as a bright pink spot. Under green light, the detectable spot is shown as a lighter brighter red spot.

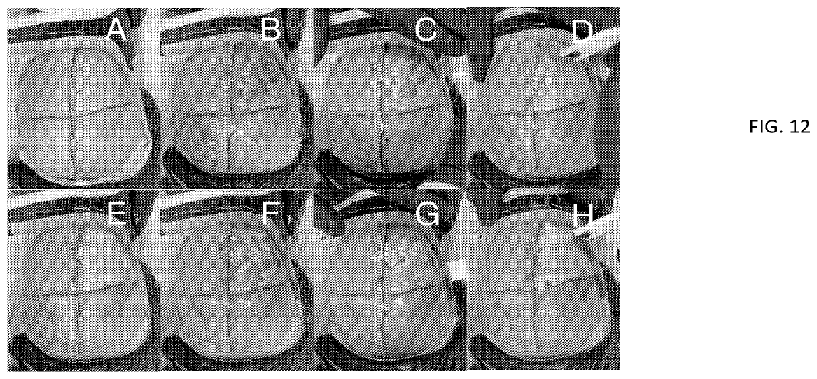

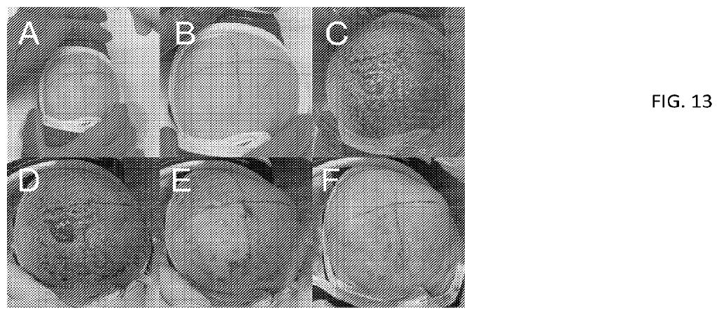

FIG. 12: Spray film was reapplied to the animal shown in FIG. 11 and pictures above show A. Spray A applied (t=−25 s) B. Spray B applied (t=−20 s) C. 20 seconds after full application of the spray film D. Scraping of the exposed quadrant E. 2^{nd} reapplication of Spray A F. 2 reapplication of Spray B G. 20 s after 2^{nd} reapplication of Spray B and H. removal of the 2^{nd} reapplication. Note that the intensity of the blue spot indicating exposure site has decreased from the original spray in FIG. 1 through the 2^{nd} reapplication of the spray.

FIG. 13 VX in IPA was applied to quadrant IV of a hairless guinea pig along with an equal volume of IPA in each remaining quadrant. Pictures are shown A. before application (t=−35 s) B. after application of Spray A (t=−25 s) C. after application of Spray B (t=0 s) D. 10 seconds after full spray application E. After removal of spray film in the exposed quadrant F. After removal of all quadrants. As shown in FIG. 13, application of the first composition (spray A) and second composition (spray B) of Example 1 produces a clearly detectable blue spot on the skin with minimal background (FIG. 13D), which can be removed (FIG. 13E) by scraping off the film at that location.

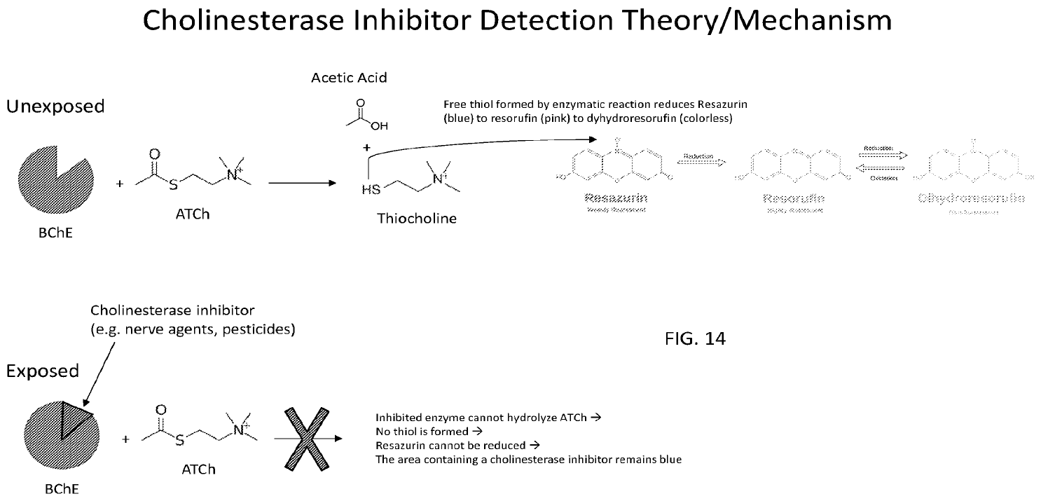

FIG. 14 provides a diagram illustrating the chemical reaction for detection of a ChE inhibitor using reaszurin in spray B. The BChE in spray A interacts with the ChE inihibitor and becomes irreversibly inhibited. In the absence of ChE, BChE in spray A reacts with ATCh to produce thiocholine and then the thiocholine interacts with resazurin to produce resorufin. Accordingly, visual indication of presence of ChE inhibitor may be given by a persistent colored spot. Visual indication of the absence of ChE inhibitor may include conversion of blue coloration to pink followed by disappearance of color. The visually detectable reaction product may include a visually detectable pink color under UV light or a white/bright spot under green light with a red-filter lens.

### Example 5 In Situ Detection of Vesicants

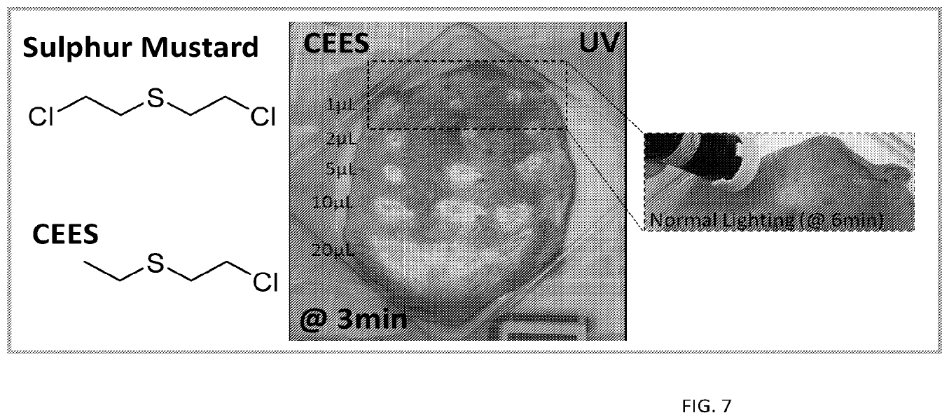

FIG. 7 shows the structures of sulfur mustard (left top) and CEES (left bottom). CEES is a less-toxic sulfur mustard simulant colloquially known as “half-mustard.” Neat CEES was spotted onto a piece of excised porcine skin in triplicate at 1, 2, 5, 10, and 20 μL and the invention embedded with the vesicant reagent chemistry such as first composition (spray A) and second composition (spray B) described in Example 2 was applied and visualized with UV light in low light conditions after 3 minutes (middle). These spots are easily visible as indicated by the bright blue spots. It should be noted that the 20 μL spots ran together and triplicates cannot be made out. The smallest volume applied, 1 μL, can be easily visualized via UV illumination under normal lighting conditions 6 minutes after spray film application (right).

### Example 6 In Situ Detection of Synthetic Opioids

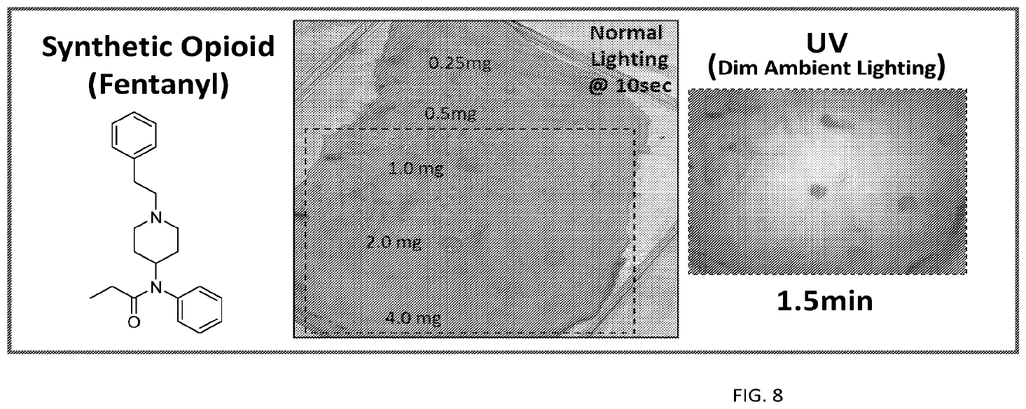

FIG. 8 shows the chemical structure of fentanyl (left). Fentanyl (0.25, 0.5, 1, 2 and 4 mg) was spotted onto a piece of excised porcine skin in duplicate and a system such as first composition (spray A) and second composition (spray B) provided in Example 3 embedded with the synthetic opioid reagent chemistry was applied. Approximately 10 s after application, spots indicating the precise location of the fentanyl is evidenced by pink spots under normal lighting conditions (middle). These spots are also easily seen as dark purple spots on a fluorescent background when viewed under UV light with dim ambient lighting (right).

What is claimed is:

1. A method to detect and at least partially decontaminate a surface of a subject suspected of being exposed to a harmful substance, the method comprising the steps of
a) applying to the surface a first composition comprising gelatin or a modified gelatin derivative, the gelatin derivative optionally comprising a hydrazide modified gelatin; and
b) applying to the surface a second composition comprising a monoaldehyde modified polysaccharide polymer
wherein steps a) and b) form a film on the surface, and wherein the first composition or second composition comprise at least one reactive agent that reacts with the suspected harmful substance to produce a visually detectable reaction product; and wherein the modified gelatin derivative is optionally modified cold-water fish gelatin (CWFG-ADH) and the monoaldehyde modified polysachharide polymer is optionally mono-aldehyde modified polygalacturonic acid (PGA-mCHO).

2. The method of claim 1, wherein the suspected harmful substance comprises a ChE inhibitor.

3. The method of claim 2, wherein the suspected harmful substance is selected from sarin, VX, Novichok, or organophosphorus nerve agents.

4. The method of claim 2 or 3, wherein the first composition comprises BChE that is inactivated/inhibited by ChE inhibitor and the second composition comprises an ATCh or other thiocholine containing substrate that is hydrolyzed by any remaining active BChE yielding the TCh product which subsequently reacts with resazurin or other suitable indicator dye initiating a color change

5. The method of claim 1, wherein the suspected harmful substance comprises a vesicant agent and the at least one reactive agent comprises luminol and an ionic liquid.

6. The method of claim 5, wherein the visually detectable reaction product fluoresces.

7. The method of claim 1, wherein the harmful substance is a synthetic opioid and the at least one reactive agent comprises Eosin Y.

8. The method of claim 7, wherein the Eosin Y complexes with a tertiary amine of the synthetic opioid to form the visually detectable reaction product.

9. The method of any of claims 1-8, further comprising removing at least a portion of the film from the surface in which the visually detectable reaction product is produced.

10. The method of claim 9, further comprising analyzing the removed portion of the film to confirm identify of the harmful substance.

11. The method of any of claims 1-10, wherein when the at least one reactive agent reacts with the suspected substance, the suspected harmful substance is at least partially neutralized.

12. The method of any of claims 1-11, wherein visually detectable reaction product develops in less than 5 minutes, or optionally, less than 1 minute.

13. A system for detecting and at least partially decontaminating a surface of a subject suspected of being exposed to a harmful substance, the system comprising
a) a first composition comprising gelatin or a modified gelatin derivative, the gelatin derivative optionally comprising a hydrazide modified gelatin; and
b) a second composition comprising a monoaldehyde modified polysaccharide polymer; and
wherein the first composition and/or second composition comprises at least one reactive agent; and
and wherein the modified gelatin derivative is optionally modified cold-water fish gelatin (CWFG-ADH) and the monoaldehyde modified polysaccharide polymer is optionally mono-aldehyde modified polygalacturonic acid (PGA-mCHO).

14. The system of claim 13, further comprising a first container in which the first composition is disposed and a second container in which the second composition is disposed.

15. The system of claim 14, wherein the first composition and/or the second composition are in a reconstitutable form, and wherein the system further comprises at least one liquid medium into which the first and/or second composition can be reconstituted.

16. The system of any of claims 13-15, wherein the suspected harmful substance comprises a ChE inhibitor.

17. The system of claim 16, wherein the suspected harmful substance is selected from sarin, VX, Novichok, and the first composition comprises BChE and the second composition comprises resazurin.

18. The system of claim 17, wherein the first composition comprises a butyrylcholinesterase enzyme, and the second composition comprises acetylthiocholine (ATCh) chloride.

19. The system of claim 17 or 18, wherein the first composition comprises butyrylcholinesterase enzyme at a concentration of about 0.50 mg/mL to about 1.0 mg/mL, CWFG-ADH at about 2 to about 10% w/v, and CaCl2) at about 5 to about 15 mM and the second composition comprises PGA-mCHO at about 1% to about 6% w/v, resazurin at about 0.1 mg/mL to about 1.0 mg/mL resazurin and ATCh chloride at about 25 mM to about 75 mM.

20. The system of any of claims 13-15, wherein the suspected harmful substance comprises a vesicant agent and the at least one reactive agent comprises luminol and an ionic liquid.

21. The system of claim 20, wherein the at least one reactive agent is in the second composition.

22. The system of claim 20 or 21, wherein the first composition comprises CWFG-ADH at about 2 to about 10% w/v, and CaCl2) at about 5 to about 15 mM and the second composition comprising PGA-mCHO at about 1% to about 6% w/v, luminol sodium salt at about 0.1 mM to about 0.9 mM and ionic liquid at about 0.5M to about 1.5 M

23. The system of any of any of claims 13-15, wherein the harmful substance is a synthetic opioid and the at least one reactive agent comprises Eosin Y.

24. The system of 23, wherein the at least one reactive agent is in the second composition.

25. The system of claim 23 or 24, wherein the first composition comprises CWFG-ADH at about 2 to about 10% w/v, and CaCl2) at about 5 to about 15 mM and the second composition comprising PGA-mCHO at about 1% to about 6% w/v, Eosin Y at about 100 microM to about 500 microM.

26. The system of any of claims 13-25, wherein the first container and/or second container comprise a manual pump mechanism.

27. The system of claim 13, wherein the first composition is loaded onto a prefabricated elastomeric film.

28. The system of any of claims 13-27, wherein the first composition further comprises water and CaCl₂, or optionally, CuCl₂ or MgCl₂, and the second composition comprises water.

29. A kit comprising a first container and second container, wherein the first and second container contain the first and second compositions of any of claims 13-28, respectively.

30. The kit of claim 29, wherein the first composition and second composition are in reconstitutable form, and wherein the kit further comprises a third container comprising a liquid medium comprising CaCl₂) and water, and optionally, a fourth container comprising a liquid medium comprising water and no CaCl₂).

31. A method to detect and at least partially decontaminate a surface of a subject suspected of being exposed to a ChE inhibitor, comprising the steps of
(a) Applying to the surface a film-forming reactive composition that visually indicates exposure to a ChE inhibitor,
(b) Applying to the surface an activating composition that reacts with the reactive composition,
wherein exposure to a ChE inhibitor is rapidly detected, the film absorbs at least some of the ChE inhibitor, and film is peelable from the skin surface.
